(19) United States
(12) Patent Application Publication
(10) Pub. No.: US 2024/0216170 A1
(43) Pub. Date: Jul. 4, 2024

(54) DRY EYE TREAMENT DEVICE

(71) Applicant: TWENTY TWENTY THERAPEUTICS LLC, South San Francisco, CA (US)

(72) Inventors: Fred Lee, South San Francisco, CA (US); Christian Gutierrez, South San Francisco, CA (US); Dimitri Azar, Chicago, IL (US)

(21) Appl. No.: 18/404,760

(22) Filed: Jan. 4, 2024

Related U.S. Application Data
(60) Provisional application No. 63/436,932, filed on Jan. 4, 2023.

(51) Int. Cl. A61F 9/00; A61K 9/00

(52) U.S. Cl. CPC A61F 9/0017; A61F 9/0026; A61K 9/0051

(57) ABSTRACT

A dry eye treatment device includes a substrate. At least one reservoir is formed in the substrate. A histamine agonist is disposed within the at least one reservoir. The histamine agonist is configured to be delivered to at least one histamine receptor on or adjacent to an eye.

### RELATED APPLICATION

This application claims priority from U.S. Provisional Application No. 63/436,932, filed 4 Jan. 2023, the subject matter of which is incorporated herein by reference in its entirety.

### TECHNICAL FIELD

This disclosure generally relates to a dry eye treatment device. This disclosure also generally relates to a dry eye treatment system comprising a dry eye treatment device and an external controller in electronic communication with an ECU of the dry eye treatment device.

### BACKGROUND

Dry eye is a condition that affects many people. Dry eye may result from the disruption of the natural tear film on the surface of the eye, and can result in ocular discomfort, visual disturbance, and a reduction in vision-related quality of life.

Dry Eye Disease (“DED”) is a clinical condition of the eye. DED is progressive in nature, and fundamentally results from insufficient tear coverage on the surface of the eye. This poor tear coverage prevents healthy gas exchange and nutrient transport for the ocular surface, promotes cellular desiccation, and creates a poor refractive surface for vision. Poor tear coverage typically results from: 1) insufficient aqueous tear production from the lacrimal glands (e.g. secondary to post-menopausal hormonal deficiency, autoimmune disease, LASIK surgery, etc.), and/or 2) excessive evaporation of aqueous tear resulting from dysfunction of the meibomian glands. Low tear volume causes a hyperosmolar environment that induces an inflamed state of the ocular surface. This inflammatory response induces apoptosis of the surface cells, which in turn prevents proper distribution of the tear film on the ocular surface so that any given tear volume is rendered less effective. This initiates a cycle where more inflammation can ensue, causing more surface cell damage, etc.

External factors that are not clinically based may also contribute to dry eye. These factors may include medications, dehydration, and environmental pollutants. Contact lenses, particularly soft contact lenses, are also known to cause or exacerbate the symptoms of dry eye. The contact lenses continually absorb water from the surface of the tear film in order to keep hydrated, leading to dryness of the eye. Dry eye can also be a symptom of the condition commonly known as “tired eye.” During extended periods of focused, intense use, such as heavy computer use and long distance driving, the eyes strain and blink less frequently, which can lead to insufficient lubrication of the eyes (i.e., dry eye).

There is a wide spectrum of treatments for dry eye. For example, U.S. patent application Ser. No. 17/391,835, filed 2 Aug. 2021 by Lee et al. and titled “OPHTHALMIC DEVICES, SYSTEMS AND METHODS FOR TREATING DRY EYE”, the subject matter of which is incorporated herein by reference in its entirety, discloses an electronic contact lens for treating dry eye. U.S. Pat. No. 11,298,537 (“the '537 patent”), issued 12 Apr. 2022 to Gutierrez and titled “NON-INVASIVE PERIOCULAR DEVICE FOR DRY-EYE TREATMENT AND CLOSED-LOOP METHODS FOR OPERATING SAME”, the subject matter of which is incorporated herein by reference in its entirety, discloses a ring-shaped periocular device/assembly/neurostimulator for providing dry-eye therapy by stimulating the lacrimal gland to stimulate tear production.

It is also known to provide therapeutic agents on or adjacent to a user's eye to treat certain eye conditions. For example, U.S. Pat. No. 11,399,976 (“the '976 patent”), issued 2 Aug. 2022 to Gutierrez and titled “EYE MOUNTED DEVICE FOR THERAPEUTIC AGENT RELEASE”, the subject matter of which is incorporated herein by reference in its entirety, discloses ophthalmic devices for treating conditions of the eye. The devices of the '976 patent provide a targeted and controlled delivery of a therapeutic agent to a treatment site of an eye. For example, the '976 patent discloses a subtarsal iontophoretic therapeutic agent delivery device(s) in which the device is placed on the conjunctiva. The '976 patent also discloses eye mountable corneal iontophoretic therapeutic agent delivery device(s).

The continued development of more and more effective ophthalmic devices for treating dry eye or various other eye conditions is beneficial due to the quality of life disruptions these eye conditions can cause to the general public.

### SUMMARY

In an aspect, alone or in combination with any other aspect, a dry eye treatment device comprises a substrate, at least one reservoir formed in the substrate, and a histamine agonist disposed within the at least one reservoir. The histamine agonist is configured to be delivered to at least one histamine receptor on or adjacent to an eye.

In an aspect, alone or in combination with any other aspect, a dry eye treatment system comprises the dry eye treatment device and an external controller. The device comprises an ECU and at least one electrode disposed on the substrate. The ECU is electrically connected to the at least one electrode. The ECU is configured to selectively cause the at least one electrode to electrically stimulate a target tissue in or adjacent to the eye. The external controller is in electronic communication with the ECU for selectively controlling the ECU.

In an aspect, alone or in combination with any other aspect, the dry eye treatment device comprises at least one controlled release mechanism adjacent the at least one reservoir. The at least one controlled release mechanism is configured to release the histamine agonist from the at least one reservoir to the at least one histamine receptor.

In an aspect, alone or in combination with any other aspect, the at least one controlled release mechanism is a polymeric layer that facilitates a passive delivery of the histamine agonist to the at least one histamine receptor.

In an aspect, alone or in combination with any other aspect, the at least one controlled release mechanism comprises a valve.

In an aspect, alone or in combination with any other aspect, the valve is a metallic film electrically connected to a power source that selectively stimulates the metallic film to release the histamine agonist.

In an aspect, alone or in combination with any other aspect, the at least one controlled release mechanism further comprises a polymeric layer overlying the metallic film. The metallic film is between the at least one reservoir and the polymeric layer such that histamine agonist released from the at least one reservoir via the metallic film egresses into the polymeric layer.

In an aspect, alone or in combination with any other aspect, the dry eye treatment device comprises an ECU electrically connected to the at least one controlled release mechanism and configured to selectively cause the at least one controlled release mechanism to release the histamine agonist from the at least one reservoir.

In an aspect, alone or in combination with any other aspect, the dry eye treatment device comprises at least one sensor for monitoring at least one eye condition. The histamine agonist is released in response to the at least one monitored eye condition.

In an aspect, alone or in combination with any other aspect, the dry eye treatment device comprises at least one electrode disposed on the substrate for selectively electrically stimulating a target tissue in or adjacent to the eye.

In an aspect, alone or in combination with any other aspect, the at least one electrode is adjacent the at least one reservoir such that the selective electrical stimulation at least partially urges the released histamine agonist into the at least one histamine receptor via iontophoresis.

In an aspect, alone or in combination with any other aspect, the dry eye treatment device comprises an overmold polymeric layer formed around substantially an entirety of the substrate. The substrate may be fully encapsulated by the overmold polymeric layer.

In an aspect, alone or in combination with any other aspect, the dry eye treatment device comprises a cap polymeric layer on or adjacent to a proximal substrate surface of the substrate. The cap polymeric layer and the substrate collectively define a holding chamber of the at least one reservoir.

In an aspect, alone or in combination with any other aspect, the dry eye treatment device comprises a support polymeric layer on or adjacent to a distal substrate surface of the substrate. The support polymeric layer at least partially defines at least one support opening in which portions of the overmold polymeric layer may be received. The at least one support opening may be aligned with the at least one reservoir such that the histamine agonist from the at least one reservoir is controllably released through the at least one support opening and into the overmold polymeric layer.

In an aspect, alone or in combination with any other aspect, the dry eye treatment device is configured to be inserted into the inferior fornix of a patient.

In an aspect, alone or in combination with any other aspect, the substrate is ring-shaped and configured to be positioned in or adjacent to the ocular fornix to at least partially encircle a portion of the eye.

In an aspect, alone or in combination with any other aspect, the dry eye treatment device is configured to be worn on the eye.

### BRIEF DESCRIPTION OF THE DRAWINGS

For a better understanding, reference may be made to the accompanying drawings, in which:

FIG. 1 schematically depicts a side view of a dry eye treatment device according to one aspect of the present invention, including the device in an example use environment;

FIG. 2 schematically depicts a rear view of the device of FIG. 1;

FIG. 3 schematically depicts a front view of the device of FIG. 1;

FIG. 4 is a cross-sectional view taken along line 4-4 in FIG. 2;

FIG. 5 a schematic depiction of a dry eye treatment system according to one aspect of the present invention;

FIG. 6 is a schematic depiction of the device of FIG. 1, including the device in an alternate configuration; and

FIG. 7 is a schematic depiction of the device of FIG. 1, including the device in an alternate configuration, including the device in an example use environment.

### DESCRIPTION OF ASPECTS OF THE DISCLOSURE

Unless defined otherwise, all technical and scientific terms used herein have the same meaning as is commonly understood by one of ordinary skill in the art to which the present disclosure pertains.

As used herein, the term “patient” can refer to any warm-blooded organism including, but not limited to, human beings, pigs, rats, mice, birds, cats, dogs, goats, sheep, horses, monkeys, apes, rabbits, cattle, farm animals, livestock, etc.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that when an element is referred to as being “on,” “connected” to, “coupled” with, “contacting,” etc., another element, it can be directly on, connected to, coupled with or contacting the other element or intervening elements may also be present. It will also be appreciated by those of skill in the art that references to a structure or feature that is disposed “adjacent” another feature may not have portions that overlap or underlie the adjacent feature.

Spatially relative terms, such as “under” and the like, may be used herein for ease of description to describe one element or feature's relationship to another element(s) or feature(s) as illustrated in the Figures. It will be understood that the spatially relative terms can encompass different orientations of a device in use or operation, in addition to the orientation depicted in the Figures. For example, if a device in the Figures is inverted, elements described as “under” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

The invention comprises, consists of, or consists essentially of the following features, in any combination.

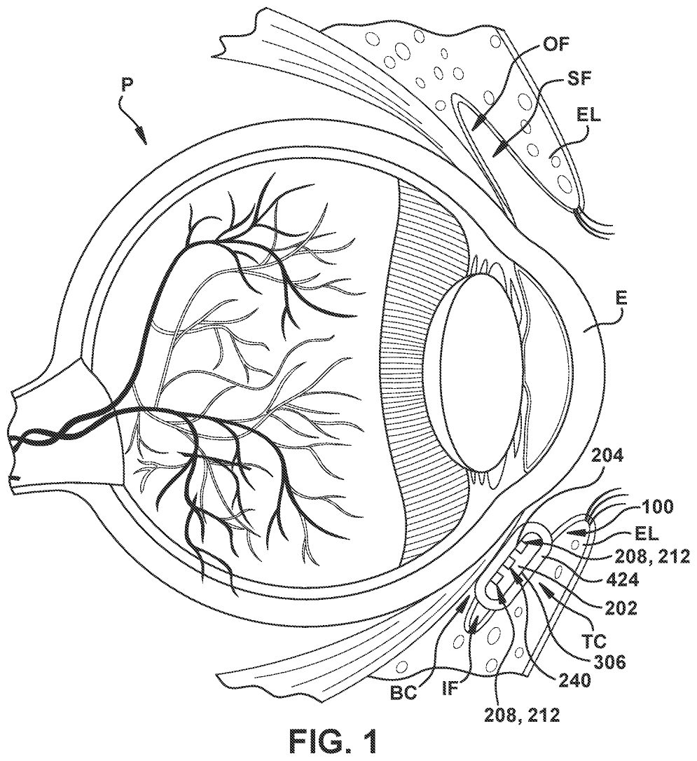

FIG. 1 depicts an example dry eye treatment device 100 designed in accordance with the present disclosure. The example device 100 of FIG. 1 is configured to be inserted into the inferior fornix IF of a patient P, though the device 100 may be configured to be inserted into the superior fornix SF, worn on a selected one of the patient's eyes E, or be positioned in other desired location adjacent the selected eye E.

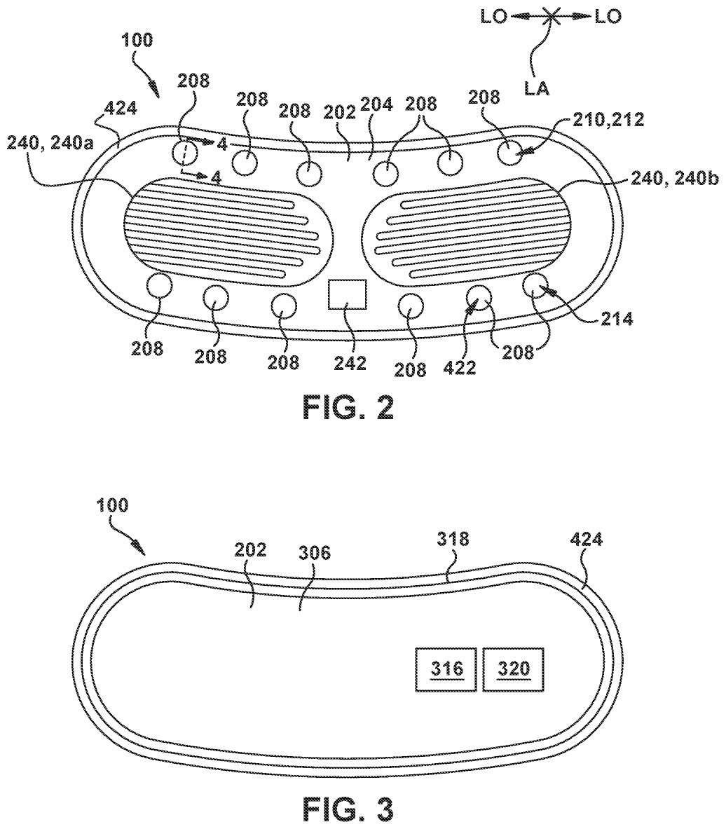

As shown in FIG. 2, the device 100 includes a substrate 202 at least partially formed of polyimide, liquid crystal polymer, parylene, polyether ether ketone, polyethylene terephthalate, poly(methyl methacrylate), polyurethane, a rigid gas permeable polymer (e.g., rigid gas permeable fluorosilicone acrylate), a silicon-based polymer, a silicone acrylate, cyclic olefin co-polymer (COP/COC), a hydrogel, any other desired material, or a combination thereof. The substrate 202 may have an average thickness (a thickness along an entire longitudinal length of the device 100) between 10 μm and 100 μm (preferably, about 80 μm) though the average thickness of the polymeric substrate may be selected as desired for specific use environments. The term “longitudinal” is used herein to indicate a substantially horizontal direction, in the orientation of FIG. 1, with the “longitudinal” direction being indicated as “LO” in FIG. 1. The “thickness” of the substrate is measured along a lateral direction. The term “lateral” is used herein to indicate a direction substantially perpendicular to the “longitudinal” direction, with the “lateral” direction being indicated as “LA” in FIG. 1.

The substrate 202 may have a shape and sufficient flexibility for mounting to the contour of the geometry of the interior fornix IF. The flexibility of the substrate 202 may be characterized based on the flexural strength or flexural modulus of the material(s) making up the substrate 202. The flexural strength of a material is its ability to resist deformation under load. For materials that deform significantly (sufficient flexibility) but do not break, the load at yield, typically measured at 5% deformation/strain of the outer surface, is reported as the flexural strength or flexural yield strength. In certain embodiments, the substrate 202 has a flexural strength or flexural yield strength of between 30 MPa and 175 MPa, preferably between 40 MPa and 130 MPa, for example about 100 MPa, and has a flexural modulus between 0.5 and 7.5 GPa, preferably between 1.0 GPa and 5.0 GPa, for example about 3 GPa, which is measured using an ASTM D70 or ISO 178 test.

As shown in FIGS. 2-3, the substrate 202 comprises a distal substrate surface 204 and a proximal substrate surface 306. As used herein, the term “proximal surface” refers to a first surface of the substrate 202, while the term “distal surface” refers to a second surface laterally opposing the first surface. For example, the distal substrate surface 204 may be configured to face toward the bulbar conjunctiva BC (FIG. 1), while the proximal substrate surface 306 may be configured to face toward the tarsal conjunctiva TC (FIG. 1).

In the example configuration shown in FIG. 2, the substrate 202 includes the one or more reservoirs 208 (here, a plurality of reservoirs 208) integrated therewith or formed therein. The reservoirs 208 may each comprise a holding chamber 210 for a therapeutic agent 212 and an egress 214 (i.e., an opening in the distal substrate surface 204) for release of the therapeutic agent 212 from the holding chamber 210. The reservoirs 208 are compatible with various physical forms of therapeutic agents 212 including aqueous (liquid), gel, dry (powder), and/or combinations thereof. In some embodiments, the reservoirs 208 provide a means for temporary storage of one or more types of therapeutic agents 212 to allow for on-demand release and delivery of the therapeutic agents 212 at a programmed time with a controlled rate. In some embodiments, each reservoir 208 holds a single type of therapeutic agent 212 (the same or different from other reservoirs 208). In other embodiments, each reservoir 208 holds multiple types of therapeutic agents 212 (the same or different from other reservoirs 208). In other embodiments, a first type of therapeutic agent 212 is disposed within a first subset of the reservoirs 208 and a second type of therapeutic agent 212 is disposed within a second subset of the reservoirs 208. Each of the reservoirs 208 may have a volume from 0.01 nL to 100 μL, and store a known quantity or volume of therapeutic agent 212.

The therapeutic agent 212 comprises any desired pharmaceutical agent or mixture of individual pharmaceutical agents configured to be delivered to a target tissue adjacent a selected one of the patient's eyes E for the treatment of one or more medical conditions of the selected eye E. In various embodiments, the therapeutic agent 212 may at least partially comprise a histamine agonist (i.e., one or more therapeutic compounds which stimulate/excite at least one histamine receptor on or adjacent to the selected eye E). The pharmacodynamics of the histamine agonist may be such that the histamine agonist, when delivered to the target tissue, produces a localized allergic response (via the excitement of the histamine receptor(s)) on or adjacent to the selected eye E. The pharmacodynamics of the histamine agonist may also be configured such that non-ocular systematic responses (i.e., responses in areas such as the patient's nose, ears, throat, etc.) do not occur or do not significantly occur in response to the delivery of the histamine agonist. While producing an allergic response in the patient via the stimulation of the histamine receptor(s) may seem contrary to typical treatment methods, this localized stimulation is configured to responsively stimulate the patient's lacrimal gland into producing tears. These produced tears may help to at least partially prevent and/or relieve dry eye.

As shown in FIG. 3, the device 100 may further include a power source 316 (which may include a battery and/or one or more capacitors), a communications device 318 (e.g., a WiFi antenna), and an electronic control unit 320 (i.e., hardware, software, or a combination thereof). In some embodiments, at least one of the power source 316, the communications device 318, and the electronics control unit (“ECU”) 320 are integrated with or formed at least partially within the substrate 202. In other embodiments, at least one of the power source 316, the communications device 318, and the ECU 320 are formed on the proximal substrate surface 306, though each of the at least one of the power source 316, the communications device 318, and the ECU 320 may be instead formed on the distal surface 204 or any other portion in or on the substrate 202. In other embodiments, the power source 316, the communications device 318, and the ECU 320 are formed on a separate (polymeric) substrate integrated with the substrate 202. In yet other embodiments, the power source 316, the communications device 318, and the ECU 320 are formed within a housing integrated with the substrate 202 and/or a separate substrate. The housing may be comprised of materials that are biocompatible such as polymers, bioceramics or bioglasses for radio frequency transparency, metals such as titanium, or any other material(s) or combinations thereof.

The communications device 318 may be connected (e.g., electrically connected) to the ECU 320 for wired or wireless communication with external devices/controllers via, for example, radiofrequency (RF) telemetry or WiFi. The power source 316 may be connected (e.g., electrically connected) to the ECU 320 to power and operate the components of the ECU 320. The connection of the ECU 320 to the power source 316 may be such that the ECU 320 is able to apply a signal or electrical current to one or more electronic components (e.g., controlled release mechanisms, electrodes, and/or sensors) connected to the ECU 320. The ECU 320 may include discrete and/or integrated electronic circuit components (e.g., one or more processors) that implement analog and/or digital circuits capable of producing the functions attributed to the device such as applying a potential to a controlled release mechanism, and/or applying a potential to one or more electrodes. In various embodiments, the ECU 320 may include software and/or electronic circuit components such as a signal generator that generates a signal causing the power source 316 and/or other circuitry to deliver a voltage, potential, current, optical signal, or ultrasonic signal to device's electronic components, a controller that determines or senses signals either received from external devices/controllers via the communications device 318 or via electrodes or sensors of the device 100, controls release and delivery parameters of the device 100, and/or causes release and delivery of the therapeutic agent 212 via the one or more reservoirs 208, and a memory with program instructions operable on by the signal generator and the controller to perform the ECU's functions.

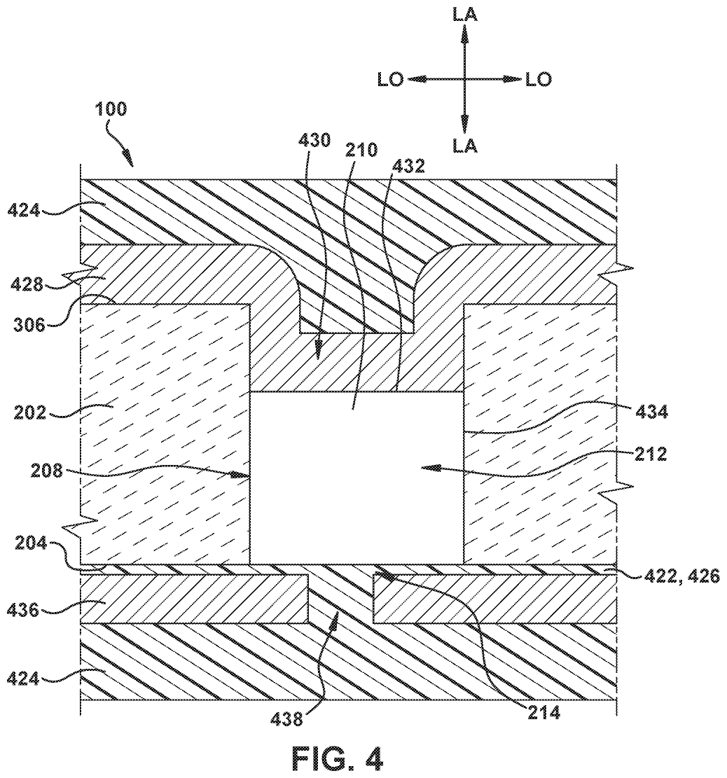

In various embodiments, the device 100 achieves release of the therapeutic agent from at least one of the reservoirs 208 to the target tissue via at least one active, passive, or combination thereof controlled release mechanism 422 (see, e.g., FIG. 4) that is located adjacent to the reservoir 208. In some embodiments, each of the reservoirs 208 comprises the holding chamber 210 for the therapeutic agent 212, the egress 214, and the active, passive, or combination thereof controlled release mechanism 422 that temporarily blocks passage of the therapeutic agent 212 from the holding chamber 210 through the egress 214. In some embodiments, a single controlled release mechanism 422 is provided for each of the reservoirs 208 (the same or different mechanism 422 provided for each reservoir 208). In other embodiments, a plurality of controlled release mechanisms 422 are provided for each of the reservoirs 208 (same or different mechanisms 422 provided for each reservoir) 208. In other embodiments, a single controlled release mechanism 422 is provided for some of the reservoirs 208, while a plurality of controlled release mechanisms 422 are provided for others of the reservoirs 208 (same or different mechanism(s) 422 provided for each reservoir 208). While the arrangement of the control release mechanism 422, reservoirs 208, and therapeutic agents 212 are described herein in particular detail with respect to several described embodiments, it should be understood that other arrangements have been contemplated without departing from the spirit and scope of the present invention. For example, different arrangements of the controlled release mechanism 422, reservoirs 208, and therapeutic agents 212 are contemplated herein such that the release and delivery of a therapeutic agent(s) 212 is targeted both temporally and spatially to a surface of a target tissue on or adjacent to a selected on of the patient's eye E (e.g., the bulbar conjunctiva BC) where optimal therapeutic agent transfer into the target tissue may occur.

In some embodiments, the controlled release mechanism 422 is passive. As used herein, “passive” means that an external stimulus is not applied to cause the opening/closing of the mechanism for release of the therapeutic agent 212. In certain embodiments, the controlled release mechanism 422 is a passive polymer device (or device constructed of a similar material). For example, a passive polymer device may be used as a part of the control release mechanism to provide controlled release of the therapeutic agent 212 in constant doses over long periods, cyclic dosage, and tunable release of both hydrophilic and hydrophobic therapeutic agents. The polymer device may be a diffusion-controlled (membrane or monolithic controlled) device, a degradable-controlled (erosion or chemically controlled) device, or a solvent-activated-controlled (swelling- or osmotically-controlled) device. In a reservoir type diffusion-controlled device, the therapeutic agent may be encapsulated or provided behind a polymer membrane (e.g., encapsulated or closed off within the reservoir by a polymer layer). Diffusion through the polymer membrane is the rate limiting step. The polymer membrane may be formed of a hydrogel (e.g., polymacon or a silicone-hydrogel), silicone ethylene-vinyl acetate copolymers, polyurethane, polyethylene, polymethylmethacrylate, polyhydroxyethylmethacrylate, a silicon-based polymer, a silicone elastomer, any other desired material, or any combination thereof. In a monolithic type diffusion-controlled device, the therapeutic agent may be distributed in a polymer matrix. For example, the therapeutic agent may be dissolved (or dispersed if the concentration exceeds the polymer's solubility limit) in a nonswellable or fully swollen matrix that does not degrade during its therapeutic life. Diffusion through the polymer membrane is the rate limiting step, in many use environments. Moreover, an environmental fluid such as tear film may leach the therapeutic agent out of the matrix if the polymer is permeable to the fluid. If a soluble additive is mixed in the polymer matrix, fluid may enter the matrix by dissolving the additive and forming interconnected channels for release of the therapeutic agent. The polymer matrix may be formed of polymethylmethacrylate, polyhydroxyethylmethacrylate, a hydrogel, a silicon-based polymer, a silicone elastomer, any other suitable material, or any combination thereof.

In a degradable-controlled device, the therapeutic agent may be encapsulated or provided behind a polymer membrane or physically immobilized in the polymer and only released by erosion of the polymer (e.g., biodegradation or chemical degradation of the polymer). This type of device may be constructed as a reservoir type device or a monolithic type device. Degradation of the polymer membrane is the rate limiting step, in many use environments. Moreover, a chemical (e.g., an agent that causes degradation of the polymer) may be bound to the polymer, and release/activation of the chemical from the polymer, e.g., hydrolytic or enzymatic cleavage of a bond (e.g., by constituents in the tear film) may ultimately cause degradation of the polymer. The degradable polymer may be formed of poly-(vinyl pyrrolidone), partially esterified copolymers of methyl vinyl ether and maleic anhydride, copolymers of lactic and glycolic acid, polyanhydrides, any other suitable material, or any combination thereof.

In a swelling-controlled device, the therapeutic agent may be dispersed or dissolved in a polymer matrix in which it is unable to diffuse to any significant extent. When the polymer matrix is placed in an environmental fluid (e.g., tear film) that is thermodynamically compatible with the polymer, the fluid is absorbed into the polymer causing it to swell. The therapeutic agent in the swollen part can then diffuse out of the device. The swellable polymer may be formed of a hydrogel, acrylamide, poly-(ethylene glycols), any other suitable material, or any combination thereof. In an osmotic-controlled device, the therapeutic agent is released from being encapsulated or behind a semi-permeable membrane with at least one egress or orifice by utilizing osmotic pressure as the driving force. In an aqueous environment (e.g., contact with a tear film), a fluid, such as water, is transported into the encapsulation or behind the semipermeable membrane by permeation. A non-extendible polymer facilitates the build-up of hydrostatic pressure, and a solution of the therapeutic agent and the fluid is urged or “pumped” out of the egress or orifice responsive to the pressure build-up. The non-extendible polymer may be formed of polymethylmethacrylate, polyhydroxyethylmethacrylate, a hydrogel, a silicon-based polymer, a silicone elastomer, any other suitable material, or any combination thereof.

In some embodiments, the controlled release mechanism 422 is active. As used herein, “active” means that an external stimulus may be applied to cause the opening/closing of the mechanism for release of the therapeutic agent 212. For example, the device 100 may achieve on-demand therapeutic agent release through electronic control of at least one valve 422 (i.e., controlled release mechanism 422) that is physically coupled to at least one of the reservoirs 208 within the device 100. In certain embodiments, a stimulus from the power source 316 (e.g., via the ECU 320) may be applied to the at least one valve 422 (the controlled release mechanism 422) to responsively open/close the at least one valve 422. A single reservoir may contain several “valves” which can be activated at selected times to increase the effective surface area available for diffusion to the target tissue. This increases the effective dose provided at a given time. Alternatively, one or more of the valves may be activated over time, thereby maintaining a constant effective therapeutic dosage level over time. It should be contemplated that multiple discrete reservoirs with valves may be implemented, each with a discrete volume of therapeutic agent for discretized bolus delivery.

The valve(s) may be single use and opened on-demand electronically to allow therapeutic agent within the reservoir(s) to pass through the valve(s) opening towards the target tissue. Alternatively, the valve(s) may be multi-use and opened/closed on-demand electronically to allow therapeutic agent within the reservoir(s) to pass through the valve opening(s) towards the target tissue. The valve opening action may initiate therapeutic agent release into thin post-device tear film located between the device and the target tissue. The distance between the valve opening and the target tissue may be filled by the tear film (<20 μm), providing a short distance for a therapeutic agent to diffuse to the target tissue. The combination of a thin tear film, device placement and preferential therapeutic agent release to the target tissue provides a quasi-static environment that promotes an increased therapeutic agent residence time (>30 minutes vs “30 seconds for topical administration) and greater availability of therapeutic agent at the target tissue, thus maximizing absorption and posterior segment bioavailability.

In certain embodiments, the controlled release mechanism 422 is an active polymer device (or device constructed of a similar material). For example, an active polymer device may be used as a part of the control release mechanism 422 to provide controlled release of the therapeutic agent 212 in constant doses over long periods, in accordance with first-order constant release profiles, or in accordance with on-demand pulsatile signals/commands. In some embodiments, the therapeutic agent may be encapsulated or provided behind a polymer membrane (e.g., encapsulated or closed off within the reservoir by a polymer layer that acts as a valve). The polymer membrane may be an environmentally-controlled device with the ability to undergo a physical or chemical behavioral change in response to an external stimulus. For example, a temperature or pH change may be used to trigger the behavioral change of the polymer but other stimuli, such as ultrasound, ionic strength, redox potential, electromagnetic radiation, and chemical or biochemical agents, may be used. Types of behavioral change can include transitions in solubility, hydrophilic-hydrophobic balance, and conformation. Upon receiving the stimuli and undergoing the behavior change, the environmentally-controlled device may release the therapeutic agent from the reservoir(s). The polymer for the environmentally-controlled device may include hydrogels, micelles, polyplexes, polymer-drug conjugates, or combinations thereof. Hydrogels are hydrophilic (co)polymeric networks capable of imbibing large amounts of water or biological fluids. Physical or covalent crosslinks may render hydrogels insoluble in water. Various hydrogels can be engineered in accordance with aspects of the present invention to respond to numerous stimuli.

In certain embodiments, the controlled release mechanism 422 is an active metal device (or device constructed of a similar material). For example, an active metal device may be used as a part of the control release mechanism 422 to provide controlled release of the therapeutic agent 212 in constant doses over long periods, in accordance with first-order constant release profiles, or in accordance with on-demand pulsatile signals/commands. In some embodiments, the therapeutic agent may be encapsulated or provided behind a metallic film (e.g., encapsulated or closed off within the reservoir by a metal layer that acts as a valve). Therapeutic agent release may be activated electronically through application of a potential or low-level voltage stimulus from the power source (e.g., via the ECU) to the metallic thin film comprising the valve. In some embodiments, the thin film forms a seal on a side of the reservoir, which may be configured to face the bulbar conjunctiva BC. The metallic film undergoes electrodissolution when a potential is applied under presence of an environmental fluid (e.g., a tear film).

In some embodiments, gold is used as the metal film material because it is easily deposited and patterned, has a low reactivity with other substances and resists spontaneous corrosion in many solutions over the entire pH range. Gold has also been shown to be a biocompatible material. However, the presence of a small amount of chloride ion, as is naturally found in tear fluid, creates an electric potential region which favors the formation of soluble gold chloride complexes. Holding the anode potential in this corrosion region between 0.8 and 1.2 V, for example at about 1.0 V, enables reproducible gold dissolution of films having a thickness of between about 50 nm and about 500 nm. Potentials below this region are too low to cause appreciable corrosion, whereas potentials above this region result in gas evolution and formation of a passivating gold oxide layer that causes corrosion to slow or stop. Other metals such as copper or titanium tend to dissolve spontaneously under these conditions or do not form soluble materials on application of an electric potential. Although gold is used in some embodiments, it should understood that other materials may be used to achieve similar electrodissolution-mediated agent release.

In some embodiments, the controlled release mechanism 422 is a combination of one or more passive devices and one or more active devices. In certain embodiments, the controlled release mechanism 422 is a passive polymer device (or device constructed of a similar material) and an active polymer or metal device. For example, an active polymer or metal device may be used as a part of the control release mechanism 422 to provide controlled release of the therapeutic agent 212 from the one or more reservoirs 208. The therapeutic agent 212 may be encapsulated or provided behind a polymeric or metallic layer (e.g., encapsulated or closed off within the reservoir by a polymeric or metallic layer that acts as a valve). Once the active polymer or metal device is opened via external stimulus, the therapeutic agent 212 may be released out of the holding chamber 210 through the egress 214 into a passive polymer device such a polymeric matrix or hydrogel. Once the therapeutic agent 212 passes through the passive polymer device (e.g., via diffusion or osmotic pump), the therapeutic agent 212 may be released and delivered to a surface of a target tissue. Alternatively, a passive polymer device may be used as a part of the control release mechanism 422 to provide controlled release of the therapeutic agent 212 from the one or more reservoirs 208. The therapeutic agent 212 may be encapsulated or provided behind a polymeric layer (e.g., encapsulated or closed off within the reservoir by a polymeric layer that acts as a valve). Once the therapeutic agent passes through the passive polymer device (e.g., via diffusion or osmotic pump), the therapeutic agent may be released out of the holding chamber through the egress into an active polymer or metal device such as encapsulated or provided behind a polymeric or metallic layer. Once the active polymer or metal device is opened via external stimulus, the therapeutic agent may be released and delivered to a surface of a target tissue (e.g., the bulbar conjunctiva BC).

As shown in FIG. 4, the device 100 may further include an overmold polymeric layer 424 formed around substantially an entirety of the substrate 202. In some embodiments, the substrate 202 is fully encapsulated by the overmold polymeric layer 424 (see, e.g., FIG. 4). The overmold polymeric layer 424 may be formed of polymethylmethacrylate, polyhydroxyethylmethacrylate, a hydrogel (e.g., polymacon or a silicon-hydrogel), a silicon-based polymer, a silicone elastomer, any other suitable material, or any combination thereof. In certain embodiments, the overmold polymeric layer 424 has a water content between 30% and 50%, for example about 38% water content.

In the embodiment shown in FIG. 4, the controlled release mechanism 422 is a combination of a metallic thin film 426 (an active metal device) and the overmold polymeric layer 424 (a polymeric passive device). The therapeutic agent 212 may be encapsulated or provided behind the metallic thin film 426 (e.g., encapsulated or closed off within the reservoir 208 by a metallic layer that acts as a valve) such that the metallic thin film 426 is laterally between the reservoir 208 and the overmold polymeric layer 424. Once the metallic thin film 426 is opened via external stimulus (e.g., provided by the power source 316 via, for example, the ECU 320) and dissolution, the therapeutic agent 212 may be released out of the holding chamber 210 of the reservoir 208 through the egress 214 into the overmold polymeric layer 424. Once the therapeutic agent 212 passes through the overmold polymeric layer 424 (e.g., via diffusion or osmotic pump), the therapeutic agent 212 may be released and delivered to a surface of the target tissue (e.g., the bulbar conjunctiva BC). This mechanism for release of the therapeutic agent 212 may be used to achieve agent release kinetics similar to passive load-and-release drug-eluting approaches albeit with a fully-programmable and customizable active release initiation.

In other embodiments, the device 100 may include exposed access points or openings in the overmold polymeric layer 424 (e.g., hydrogel), which exposes a surface of the reservoirs 208. In these embodiments, the post-device tear film or tissue is in direct contact with the controlled release mechanism 422 or the egress 214 of the reservoir 208. Upon release of the therapeutic agent 212 from the holding chamber 210, the therapeutic agent 212 permeates directly into the post-device tear film or tissue. This mechanism for release may be used to achieve alternative release kinetics with fully-programmable and customizable active release similar to topical application of eye drops however with the benefit of drastically increased residence times, increased bioavailability and minimal drug loss. More generally, the device 100 enables customized delivery profiles which is currently unavailable with either topical eye drops or intravitreal needle injection. Advantageously, where the therapeutic window changes or is cyclic (e.g., due to circadian rhythm such as in glaucoma), the device is able to meet these changes in a fully customized manner.

As shown in FIG. 4, the device 100 may include one or more layers in addition to the substrate 202 and the overmold polymeric layer 424. For example, a cap polymeric layer 428 may be provided on or adjacent to the proximal substrate surface 306 (via, e.g., vacuum depositing). The cap polymeric layer 428 and the substrate 202 may collectively define the holding chamber 210. In particular, the cap polymeric layer 428 may include a plurality of projecting portions 430 (i.e., portions of the cap polymeric layer 428 that extend further in the lateral direction toward the distal substrate surface 204 than other portions of the cap polymeric layer 428). The projecting portions 430 of the cap polymeric layer 428 may define end walls 432 of the holding chambers 210, while the substrate 202 defines one or more side walls 434 of the holding chambers 210. The cap polymeric layer 428 may be formed at least partially from parylene (e.g., parylene C) and/or any other desired material.

The device 100 may also include a support polymeric layer 436 provided on or adjacent to the distal substrate surface 204 (via, e.g., vacuum depositing). The support polymeric layer 436 may help define at least one support opening 438 in which portions of the overmold polymeric layer 424 may be received. Each support opening 438 may be aligned with an associated one of the reservoirs 208 such that the therapeutic agent 212 from the reservoirs 208 may be controllably released through the support openings 438 and into the overmold polymeric layer 424. Each support opening 438 thus defines an area in which the therapeutic agent may be released into overmold polymeric layer 424 from an associated reservoir 208. The flow (in terms of both speed and location) of the therapeutic agent 212 from the reservoirs 208 into the overmold polymeric layer 424 may be configured by adjusting/selecting the release areas formed by the support openings 438. It should be understood that the support openings 438 may provide similar functionality even when the overmold polymeric layer 424 is not provided laterally between the reservoirs 208 and the target tissue. The support polymeric layer 436 may be formed at least partially from parylene (e.g., parylene C) and/or any other desired material.

In some embodiments, the device 100 may also include at least one layer of epoxy for retaining at least the electrical components of the device 100 connected to the substrate 202. Furthermore, in some embodiments, at least one of the holding chambers 210 may be lined with a layer of polymeric material and/or a coating. The coating may be a passive, hermetic, insulator, and/or inert coating such as a dielectric. The layer of polymeric material and/or the coating may be formed from an approved agent-contacting material. The material(s) chosen for the coating/layer of polymeric material may be therapeutic agent specific. Therefore, the material for the coating/therapeutic agent, when provided, may be selected based on the therapeutic agent 212 being a histamine agonist.

Returning to FIG. 1, the device 100 is positioned such that the bulbar conjunctiva BC is the target tissue to which the therapeutic agent 212 is configured to be delivered. When the therapeutic agent 212 comprises a histamine agonist, the histamine agonist is delivered to at least one histamine receptor located in the bulbar conjunctiva BC upon release, which in turn excites/stimulates the at least one histamine receptor. The stimulation of the histamine receptor(s) in the bulbar conjunctiva BC responsively excites/stimulates an adjacent lacrimal gland of the patient (e.g., through the trigeminal ganglion) to produce tears in the patient's selected eye E. This tearing at least partially helps to prevent and/or relieve dryness in the selected eye E.

In the above, therapeutic agent penetration across the target tissue has been provided by passive diffusion alone. However, some embodiments of the device 100 may be configured such that the therapeutic agent penetration may selectively be greatly increased beyond passive diffusion alone by means of an external energy source, in particular by iontophoresis. Iontophoresis is a local non-invasive technique in which an electric field is applied to enhance ionized therapeutic agent penetration into tissue. The '976 patent, for example, discusses the use of iontophoresis to facility/enhance the delivery of a therapeutic agent into a target tissue.

As shown in the embodiment of FIG. 2, the device 100 may include at least one electrode 240 (shown here as a plurality of electrodes 240) that selectively electrically stimulates the target tissue (i.e., provides an external energy source in the form of current/electricity to the target tissue) at least for the delivery of the therapeutic agent 212 into the target tissue via iontophoresis. When the therapeutic agent 212 is a histamine agonist, for example, the electrical stimulation selectively provided by at least one of the electrodes 240 at least partially urges the histamine agonist into at least one histamine receptor in the bulbar conjunctiva BC via iontophoresis. The electrodes 240 are connected electrically to the ECU 320 to be selectively activated/energized via a voltage/potential/current from the ECU 320 or from the power source 316 upon instruction from the ECU 320. The length and/or strength of the electrical stimulation may be programed into the ECU 320 as desired.

Similar to the electrodes of the '976 patent, at least one of the electrodes 240 (shown here as a first electrode 240a) may be an anode used for transporting the therapeutic agent 212 into the target tissue via electromigration, while at least one other of the electrodes 240 (shown here as a second electrode 240b) may be a cathode for maintaining electroneutrality within the target tissue. However, in some embodiments, each the first and second electrodes 240a, 240b may be used for electromigration. In other embodiments, at least one of the electrodes 240 may include both anode and cathode portions providing both iontophoresis and electroneutrality functionality.

At least one of the electrodes 240 may be disposed on the substrate and located adjacent to at least one of the reservoirs 208 so that the therapeutic agent 212 released from the adjacent reservoir(s) 208 may be at least partially electromigrated into the target tissue. In the embodiment shown in FIG. 2, each of the first and second electrodes 240a, 240b are located adjacent to a plurality of reservoirs 208. However, the electrodes 240 and the reservoirs 208 may be arranged on the substrate 202 in any desired manner. In some embodiments, the electrode-reservoir arrangement of the device 100 may be similar to any of the electrode-reservoir arrangements shown/discussed in the '976 patent. For example, the electrode-reservoir arrangement may be such that at least one of the electrodes 240 is disposed “under” at least one reservoir 208 such that the electrode is adjacent to or defines at least a portion of the end wall 432, or such that the first and second electrodes 240a, 240b are at opposing longitudinal ends of the substrate 202 while at least one of the reservoirs 208 is positioned longitudinally between the first and second electrodes 240a, 240b.

In some embodiments, the electrodes 240 may provide functionality in addition to their ionotropic/electromigration functionality. For example, the electrical stimulation provided by at least one of the electrodes 240 may be configured to stimulate ocular nerves in or adjacent to the target tissue (e.g., the bulbar conjunctiva BC) to cause tearing. In other words, the electrical stimulation selectively applied by the electrodes 240 to the target tissue's optical nerves may responsively excite/stimulate an adjacent lacrimal gland of the patient P (e.g., through the trigeminal ganglion) into producing tears. Therefore, the electrodes 240, in addition to provided electromigration functionality, may serve as an electroceutical source of tearing. In some embodiments, the electrodes 240 thus may be configured to work with the therapeutic agent 212 to at least partially cause the tearing in the patient's selected eye E. In other embodiments, the device 100 may be configured such that only the electrodes 240 serve as a source of tearing, while the reservoirs/therapeutic agent 208/212 is/are provided for a different therapeutic purpose or not provided at all.

The electrodes 240 may also but also serve to at least partially help distract the localized somatic side effects of interrogating the histamine receptor(s), which in some cases may produce localized itching. The electrical stimulation may also at least partially help relieve the patient P of itching sensations.

In some embodiments, such as the embodiment shown in FIG. 2, the device 100 may include one or more sensors 242 provided on the substrate 202 and electrically connected to the ECU 320. The sensor(s) 242 is configured to monitor one or more eye conditions, generate one or more output signals indicative of the monitored eye condition(s), and electrically transmit the output signal(s) to the ECU 320. The ECU 320, in response to receiving the output signal(s), may control the electrodes 240 and/or the controlled release mechanism(s) 422 in accordance with the monitored eye condition(s). For example, if the conditions/parameters monitored by the sensor(s) 242 indicates that the patient's eye E is dry or drying, the ECU 320 may be actuated to cause tear production in one more of the manners described above (releasing the therapeutic agent 212 and/or providing electrical stimulation). The ECU 320 may also be configured to store the received monitored conditions/parameters to be used by the patient P or a medical professional for determining, monitoring, and/or adjusting a treatment plan.

In some embodiments, the sensor(s) 242 may include a reservoir release sensor that is configured to monitor one or more parameters, such as, but not limited to, the opening progress of at least one controlled release mechanism 422, the rate at which the monitored controlled release mechanism 422 opens, and when the opening is completed. The monitored parameters may be used by the ECU 320 may be used to determine if the monitored controlled release mechanism(s) 422 are functioning as desired and the therapeutic agent 212 is being released in a desired manner.

In some embodiments, the sensor(s) 242 may include one or more tear film sensors. The tear film break-up sensors are configured to monitor one or more tear-film dynamic factors, such as, but not limited to, tear-film break-up time, rate of evaporation, and total available volume. The monitored tear-film dynamic factors may be useful in determining how dry the selected eye E is or how quickly the selected eye E is drying.

In some embodiments, the sensor(s) 242 may include a pH sensor that monitors tear pH levels, as tear osmolality and pH have been shown to correlate with dry eyes.

In some embodiments, the sensor(s) 242 may include a blink sensor configured to monitor the blink rate of the patient P. Normal individuals display interblink times on average 4±2 seconds, while patients with dry eye may display significantly decreased times averaging 1.5±0.9 seconds in an attempt to maximize the tear supply to the ocular surface. Thus, blink rate can be used to help identify a dry eye condition.

In some embodiments, the sensor(s) 242 may include a chlorine sensor configured to an ion sensor configured to measure the concentration levels of the major ions, such as, but not limited to, Na+, K+, and Cl−, in the tear fluid film. Relatively high tear film K+ has been suggested to be important for the health of the ocular surface epithelium. More generally, lacrimal gland fluid secretion is an important determinant of tear film ionic composition, for example, fluid produced by the lacrimal gland is enriched in K+ and Cl− relative to serum. Therefore, monitoring the ionic content of the tear fluid is an important factor to management of ocular surface health.

In some embodiments, the sensor(s) 242 may include one or more dosage sensors. Each dosage sensor may be an electrochemical sensors that is tuned to the therapeutic agent(s) 212 within the reservoir(s) 208, and can detect rate and concentration of therapeutic agent release. In some embodiments, the sensor(s) 232 may include any number of the sensors described above or any other desired sensor.

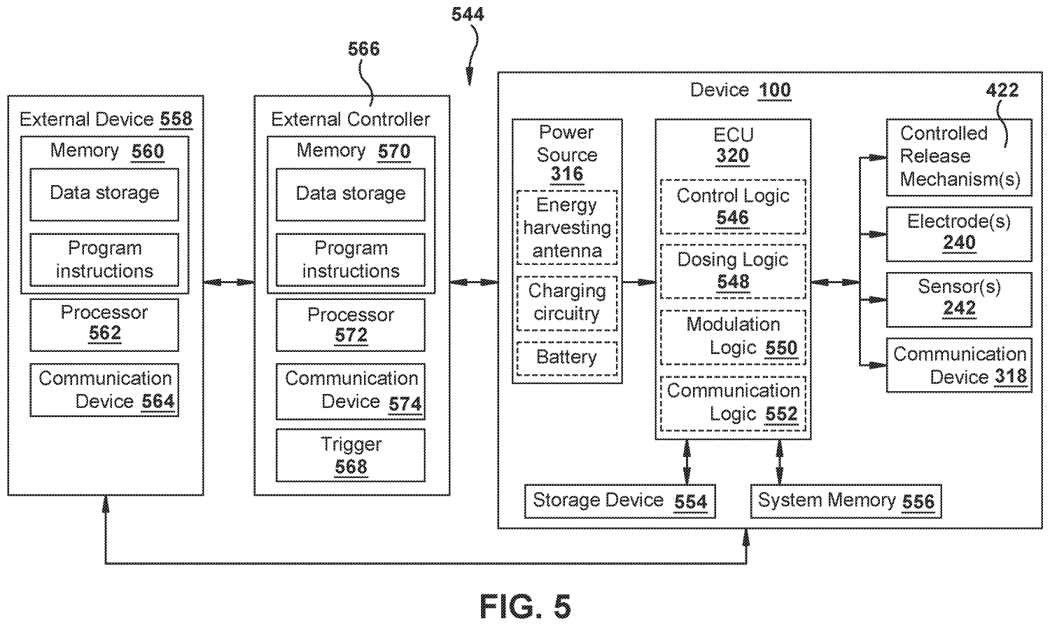

As shown in FIG. 5, the device 100 may be part of a dry eye treatment system 544. In certain embodiments, the device 100 may be disposed in a selected one or both eyes E of the patient P. The substrate 202 includes software and/or electronic circuit components that provide therapeutic agent delivery and/or electric stimulation. The software and/or electronic circuit components includes at least one of the power source 316, the ECU 320, the controlled release mechanism(s), the electrode(s) 240, the sensor(s) 242, and the communications device 318.

In certain embodiments, the ECU 320 includes one or more conventional processors, microprocessors, or specialized dedicated processors that include processing circuitry operative to interpret and execute computer readable program instructions, such as program instructions for controlling the operation and performance of one or more of the various other components of device 100 for implementing the functionality, steps, and/or performance of the present embodiments. In certain embodiments, the ECU 320 interprets and executes the processes, steps, functions, and/or operations of the present invention, which may be operatively implemented by the computer readable program 548. For example, the ECU 320 includes control logic 546, dosing logic 548, modulation logic 550, and communication logic 552 that communicate interactively with the reservoirs 208, the electrodes 240, the sensor(s) 242, and the communications device 318. In some embodiments, the information obtained or generated by the ECU 320, e.g., the status of agent delivery, agent dosages, temporal location in therapeutic window, etc., can be stored in a storage device 554.

The storage device 554 may include removable/non-removable, volatile/non-volatile computer readable media, such as, but not limited to, non-transitory machine readable storage medium such as magnetic and/or optical recording media and their corresponding drives. The drives and their associated computer readable media provide for storage of computer readable program instructions, data structures, program modules and other data for operation of the ECU 320 in accordance with the different aspects of the present invention. In some embodiments, the storage device 554 stores an operating system, application programs, and program data.

A system memory 556 may include one or more storage mediums, including for example, non-transitory machine readable storage medium such as flash memory, permanent memory such as read-only memory (“ROM”), semi-permanent memory such as random access memory (“RAM”), any other suitable type of non-transitory storage component, or any combination thereof. In some embodiments, an input/output system (BIOS) including the basic routines that help to transfer information between the various other components of device 100, such as during start-up, may be stored in the ROM. Additionally, data and/or program modules, such as at least a portion of operating system, program modules, application programs, and/or program data, that are accessible to and/or presently being operated on by one or more processors, may be contained in the RAM. In embodiments, the program modules and/or application programs can comprise, for example, control logic 546, dosing logic 548, modulation logic 550, and communication logic 552, which provides the instructions for execution of the one or more processors.

The communication device 318 may include any transceiver-like mechanism (e.g., a network interface, a network adapter, a modem, or combinations thereof) that enables device 100 to communicate with remote devices or systems, such as a mobile device or other computing devices such as, for example, a server in a networked environment, e.g., cloud environment. For example, device 100 may be connected to remote devices or systems via one or more local area networks (LAN) and/or one or more wide area networks (WAN) using communication device 318.

The ECU 320 can be remotely accessed following implant through an external device 558, such as a smartphone or any other portable or non-portable computing device. For example, the external device 558 can be used by the patient P and/or a healthcare professional to check and program the ECU 320 before or after distribution to the patient P (e.g., while the patient P is wearing the device 100), adjust release and delivery parameters during a delivery process, e.g., providing an initial set of the release and delivery parameters, and read any data concerning dosage, delivery, and compliance of the device 100 during or after a dosing regimen. The external device 558 may also be configured to monitor at least one eye condition, monitor histamine agonist dosage, and/or monitor histamine agonist delivery. In some embodiments, the external device 558 may be configured to schedule reminders and/or access local or hyper-local weather and air quality data. In some embodiments, the external device 558 comprises a memory 560 (e.g., a storage device or system memory), one or more processors 562, and a communications device 564 such as a WiFi antenna. The external device 558 may communicate with the ECU 320 via wired or wireless communication methods, such as, e.g., wireless radio frequency transmission.

The ECU 320 may also or instead be remotely accessed following implant through an external controller 566. The external controller 566 may include a trigger 568 that, when actuated (e.g., physically manipulated, such as by being depressed) by the patient P and/or a medical professional, instructs the ECU 320 to deliver a predetermined amount of the therapeutic agent 212 to the target tissue (via actuation of the controlled release mechanism(s) 422) and/or to electrically stimulate the target tissue (via energization of at least one of the electrodes 240). Therefore, via the interaction between the external controller 566 and the device 100, tears in the selected eye may be produced on-demand responsive to actuation of the external controller 566 to at least partially treat the patient's dryness in the selected eye E. In some embodiments, the external controller 566 comprises a memory 570 (e.g., a storage device or system memory), one or more processors 572, and a communications device 574 such as a WiFi antenna. The external controller 566 may communicate with the ECU 320 via wired or wireless communication methods, such as, e.g., wireless radio frequency transmission.

The external controller 566 may also communicate with the external device 558 via wired or wireless communication methods, such as, e.g., wireless radio frequency transmission. Usage statistics of the external controller 566 (e.g., the number of times and/or frequency at which the user operates the device 100 via the external controller 566) may be transmitted to the external device 558 to be stored and monitored.

In some of the embodiments of the system 544, the “trigger” functionality of the external controller 566 may also be incorporated into the external device 558. In such embodiments, the external controller 566 may be provided for “trigger” redundancy purposes or may be omitted from the system 544.

As discussed herein, the system 544 may be configured to control release of the therapeutic agent 212 from the reservoirs 208, and control application of a potential to one or more electrodes 240 that causes electromigration of the therapeutic agent 212 into a target tissue and/or stimulates optical nerves in the target tissue. In particular, the device 100 may perform tasks (e.g., process, steps, methods and/or functionality) in response to the ECU 320 executing program instructions contained in non-transitory machine readable storage medium, such as the system memory 556. The program instructions may be read into the system memory 556 from another computer readable medium (e.g., non-transitory machine readable storage medium), such as the storage device 554, or from another device such as the external device 558, external controller 566, and/or server within or outside of a cloud environment. In additional or alternative embodiments, hardwired circuitry may be used in place of or in combination with the program instructions to implement the tasks, e.g., steps, methods and/or functionality, consistent with the different aspects. Thus, the steps, methods and/or functionality disclosed herein can be implemented in any combination of hardware circuitry and software.

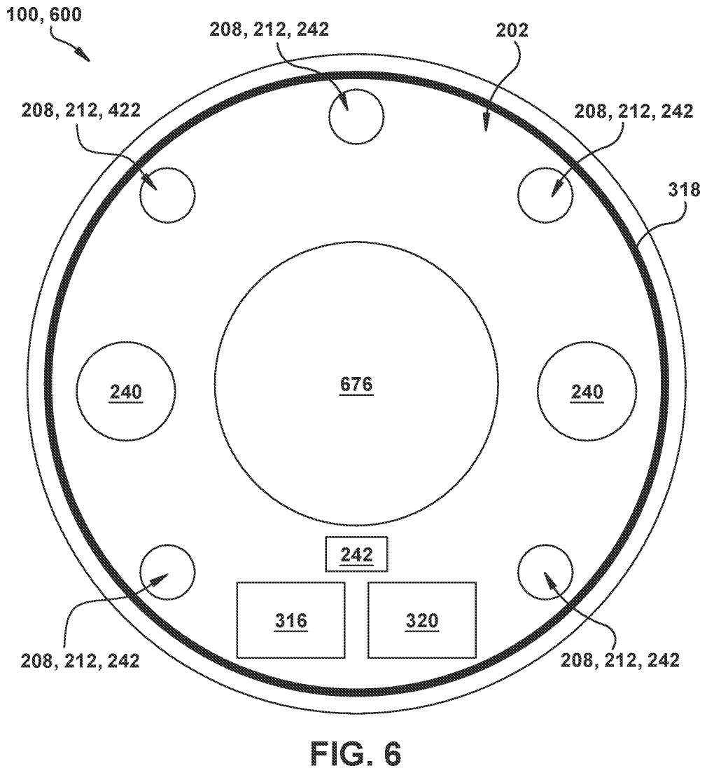

While various embodiments are disclosed herein with respect to device that is mounted under a patient eyelid (e.g., in the inferior fornix IF), this is not intended to be restrictive. For example, as shown in FIG. 6, any embodiment of the device 100 (shown here as the device 600) may be configured to be worn on a selected one of the patient's eyes E. In other words, the device 600 may be configured to fit discreetly over at least a portion of the corneal surface such that the device 600 does not block or affect vision in any way (via a visual clearance area 676) and is compatible with standard contact lens materials while maintaining desired contact with the cornea for delivering the therapeutic agent 212 to (via the controlled release mechanism(s) 422 and the one or more reservoirs 208) and/or electrically stimulating (via the one or more electrodes 240) a target tissue on the patient's selected eye E. The target tissue for the embodiment of FIG. 6 may be the anterior segment of the selected eye E. The anterior segment or anterior cavity is the front third of the selected eye E that includes the structures in front of the vitreous humor: the cornea, iris, ciliary body, and lens.

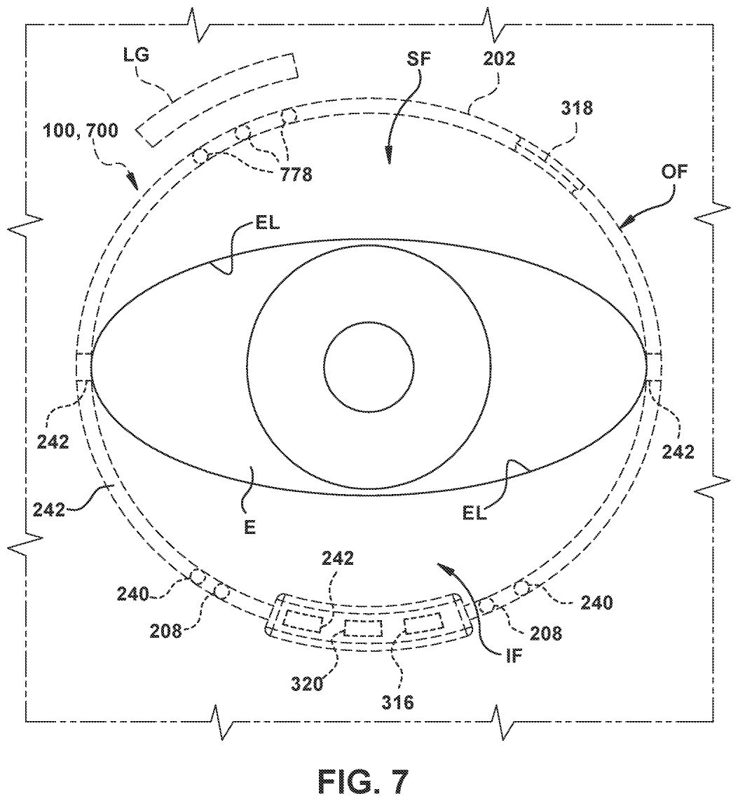

Furthermore, as shown in FIG. 7, any embodiment of the device 100 (shown here as the device 700) may be configured to be positioned in or adjacent to the ocular fornix OF under the patient's eyelids EL to at least partially encircle a portion of the eye E. As such, the substrate 202 and the device 700 as a whole may be modified into a ring-shape similar to that of the device taught by the '537 patent and/or to include any of the features and/or operating characteristics disclosed in the '537 patent.

The reservoirs 208 and the electrodes 240 of the device 700 may be positioned on the substrate 202 such that they are located in the inferior fornix IF similar to that of the device 100 of FIGS. 1-4. In some embodiments, the device 700 may also include one or more secondary electrodes 778 (shown here as a plurality of secondary electrodes 778) in the superior fornix SF adjacent the lacrimal gland LG for selectively electrically stimulating the lacrimal gland LG. In some embodiments, at least one of the secondary electrodes 778 is or includes an anode for selectively electrically stimulating the lacrimal gland LG, while at least one other of the secondary electrodes 778 is or includes a cathode for maintaining electroneutrality. The secondary electrodes 778 may be electrically connected to the ECU 320 and operated in a similar manner as the electrodes 240.

Although the device(s) 100, 600, 700 and the system 544 have been largely described as treating dry eye, the device(s) 100, 600, 700 and/or the system 544 may be configured to at least partially help treat various other eye conditions via therapeutic agent delivery and/or electrical stimulation.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages may be obtained from a study of the drawings, the disclosure, and the appended claims.

We claim:

1. A dry eye treatment device, comprising:
a substrate;
at least one reservoir formed in the substrate; and
a histamine agonist disposed within the at least one reservoir, the histamine agonist being configured to be delivered to at least one histamine receptor on or adjacent to an eye.

2. The device of claim 1, further comprising at least one controlled release mechanism adjacent the at least one reservoir, the at least one controlled release mechanism being configured to release the histamine agonist from the at least one reservoir to the at least one histamine receptor.

3. The device of claim 2, wherein the at least one controlled release mechanism comprises a valve.

4. The device of claim 3, wherein the valve is a metallic film electrically connected to a power source that selectively stimulates the metallic film to release the histamine agonist.

5. The device of claim 4, wherein the at least one controlled release mechanism further comprises a polymeric layer overlying the metallic film, the metallic film being between the at least one reservoir and the polymeric layer such that histamine agonist released from the at least one reservoir via the metallic film egresses into the polymeric layer.

6. The device of claim 5, wherein the polymeric layer is formed of at least one of polymethylmethacrylate, polyhydroxyethylmethacrylate, a hydrogel, a silicon-based polymer, a silicone elastomer, and a rigid gas permeable polymer.

7. The device of claim 2, further comprising at least one sensor for monitoring at least one eye condition, the histamine agonist being released in response to the at least one monitored eye condition.

8. The device of claim 2, wherein the at least one controlled release mechanism is a polymeric layer that facilitates a passive delivery of the histamine agonist to the at least one histamine receptor.

9. The device of claim 2, further comprising an ECU electrically connected to the at least one controlled release mechanism and configured to selectively cause the at least one controlled release mechanism to release the histamine agonist from the at least one reservoir.

10. The device of claim 1, further comprising at least one electrode disposed on the substrate for selectively electrically stimulating a target tissue in or adjacent to the eye.

11. The device of claim 10, wherein the at least one electrode is adjacent the at least one reservoir such that the selective electrical stimulation at least partially urges the released histamine agonist into the at least one histamine receptor via iontophoresis.

12. The device of claim 10, further comprising an ECU configured to selectively cause the at least one electrode to electrically stimulate a target tissue in or adjacent to the eye.

13. The device of claim 1, further comprising at least one sensor for monitoring at least one eye condition.

14. A dry eye treatment system, comprising:
the device of claim 1, the device comprising an ECU and at least one electrode disposed on the substrate, the ECU being electrically connected to the at least one electrode, the ECU being configured to selectively cause the at least one electrode to electrically stimulate a target tissue in or adjacent to the eye; and
an external controller in electronic communication with the ECU for selectively controlling the ECU.

15. The system of claim 12, wherein the external controller includes a trigger via which the ECU is commanded to cause the at least one electrode to electrically stimulate a target tissue in or adjacent to the eye.

16. The system of claim 12, wherein the at least one electrode is adjacent to the at least one reservoir such that the selective electrical stimulation at least partially urges the released histamine agonist into the at least one histamine receptor via iontophoresis.

17. The system of claim 12, further comprising an external device electrically connected to at least one of the external controller and the ECU, the external device being configured for:
adjusting operating characteristics of at least one of the external controller and the ECU;
monitoring at least one eye condition;
monitoring histamine agonist dosage; and/or
monitoring histamine agonist delivery.

18. The device of claim 1, wherein the device is configured to be inserted into the inferior fornix of a patient.

19. The device of claim 1, wherein the substrate is ring-shaped and configured to be positioned in or adjacent to the ocular fornix to at least partially encircle a portion of the eye.

20. The device of claim 1, wherein the device is configured to be worn on the eye.
